(19) United States
(12) Patent Application Publication
(10) Pub. No.: US 2026/0337643 A1
(43) Pub. Date: Oct. 29, 2026

(54) METHOD FOR TREATING A LIGNOCELLULOSIC BIOMASS

(71) Applicant: IFP ENERGIES NOUVELLES, RUEIL-MALMAISON (FR)

(72) Inventors: Matthieu ARAGONES, Rueil-Malmaison Cedex (FR); Sophie COUDERC, Rueil-Malmaison Cedex (FR); Gilles FERSCHNEIDER, Rueil-Malmaison Cedex (FR)

(73) Assignee: IFP ENERGIES NOUVELLES, RUEIL-MALMAISON (FR)

(21) Appl. No.: 19/118,289

(22) PCT Filed: Sep. 27, 2023
(86) PCT No.: PCT/EP2023/076795

§ 371 Date: Apr. 3, 2025

(30) Foreign Application Priority Data
Oct. 4, 2022 (FR) 2210151

(51) Int. Cl. C12P 7/10; C12M 1/00; C12P 19/14; C13K 1/02

(52) U.S. Cl. CPC C12P 7/10; C12M 21/12; C12M 43/04; C12P 19/14; C13K 1/02; C12P 2201/00

(57) ABSTRACT

The invention relates to a process for treating a lignocellulosic biomass, comprising:
a) a step of impregnating the biomass with a liquor to obtain an impregnated biomass,
b) a step of cooking the impregnated biomass, optionally accompanied by a steam explosion, to obtain a pretreated biomass,
c) a step of enzymatic hydrolysis of the pretreated biomass, to obtain a hydrolyzed biomass in the form of sugar(s),
d) a step of solid/liquid separation of the hydrolyzed biomass in the form of sugar(s) or of the hydrolyzed biomass in the form of sugar(s) subsequently treated in one or more other steps after the enzymatic hydrolysis step c), in order to obtain a separated juice and an unconverted solid residue,
e) a step of recycling at least one portion of said unconverted solid residue to the impregnation step a) and/or to the cooking step b).

### TECHNICAL FIELD

The invention relates to a process for treating lignocellulosic biomass for producing “second-generation” (2G) sugar juices. These sugar juices may be used to produce other products via a biochemical pathway, notably by fermentation (for example alcohols such as ethanol and butanol, or other molecules, for example solvents such as acetone, etc.).

### PRIOR ART

Lignocellulosic biomass represents one of the most abundant renewable resources on Earth. The substrates considered are very varied, they relate both to ligneous substrates such as various woods (hardwoods and softwoods), coproducts derived from agriculture (wheat straw, corn cobs, etc.) or from other agrifood, paper, lignocellulosic waste, etc. industries.

The process for treating lignocellulosic biomass generally comprises
a pretreatment of the biomass by cooking, optionally coupled to a steam explosion and generally preceded by an impregnation of the biomass with an acidic, basic, neutral or oxidizing liquor,
an enzymatic hydrolysis, leading to the production of sugar juices, generally based on C5 and C6 sugars (i.e. sugars with 5 or 6 carbons),
and optionally a fermentation of these sugars by a yeast, to convert them to ethanol-type alcohol. The process also includes steps of separating and/or purifying the targeted end product (sugar, alcohol, solvent, etc.).

Lignocellulosic biomass is composed of three main polymers: cellulose (35% to 50% by weight), which is a polysaccharide essentially consisting of hexoses; hemicellulose (20% to 40% by weight), which is a polysaccharide essentially consisting of pentoses; and lignin (10% to 30% by weight), which is a polymer of complex structure and of high molecular weight, composed of aromatic alcohols connected via ether bonds. These various molecules are responsible for the intrinsic properties of plant walls and are organized in a complex entanglement. Among the three base polymers that make up lignocellulosic biomass, cellulose and hemicellulose are the ones that enable the production of 2G sugar juices.

Usually, hemicellulose is predominantly broken down into sugar during the pretreatment, and cellulose is converted into sugar (glucose) by enzymatic hydrolysis. However, crude cellulose remains difficult for enzymes to access, hence the need for a pretreatment. This pretreatment makes it possible to modify the physicochemical properties of the lignocellulosic biomass so as to improve the accessibility of the cellulose to enzymes and its reactivity to enzymatic hydrolysis.

There are many advantageous technologies for performing this pretreatment, which will be combined below under the generic term “cooking”: acidic cooking, alkaline cooking, cooking by auto-hydrolysis, steam explosion, and “organosolv pulping” processes. The latter process concerns pretreatment in the presence of one or more organic solvents and generally water. The solvent may be an alcohol (ethanol), an acid such as acetic acid or formic acid, or else acetone. “Organosolv pulping” processes lead to at least partial dissolution of the lignin and partial dissolution of the hemicelluloses. There are thus two outlet streams: the pretreated substrate with residual cellulose, hemicellulose and lignin, and the solvent phase which contains the dissolved lignin and a portion of the hemicelluloses. There is generally a step of regeneration of the solvent, which makes it possible to extract a lignin stream. Certain “organosolv pulping” treatments (notably with ethanol) are coupled with the addition of a strong acid (such as H₂SO₄). It may also be envisaged to place the biomass in contact with the solvent via an impregnation reactor before the cooking phase or to place the biomass in contact with the acid catalyst before performing “organosolv pulping” cooking.

Various configurations are reported, for example, in the publication “Production of bioethanol from lignocellulosic materials via the biochemical pathway: A review”, M. Balat, Energy Conversion and Management 52 (2011) 858-875, or in the publication “Bioethanol production from agricultural wastes: An overview”, N. Sarkar, S. Kumar Ghosh, S. Bannerjee, K. Aikat, Renewable Energy 37 (2012) 19-27.

One of the most effective pretreatments is steam explosion, notably under acidic conditions, which enables almost complete hydrolysis of hemicellulose and a significant improvement in the accessibility and reactivity of cellulose to enzymes. This pretreatment may be preceded by other treatment(s).

U.S. Pat. Nos. 8,057,639 and 8,512,512 propose a process comprising a first step of hydrolysis of hemicellulose to C5 sugars under mild conditions which thus protect them from degradation. This step is performed in a first reactor at a pressure of 1.5 bar (0.15 MPa) or more, by injection of steam, at a temperature of 110 °C or more, and optionally in the presence of a weak acid. After this step, washing is performed in order to extract and recover the sugar juices obtained from hemicellulose (generally juices of C5 sugars and C6 sugars, the relative proportion of which depends, in particular, on the nature of the biomass) before sending the remaining biomass, enriched in cellulose and lignin, to a second step (second reactor) where the steam explosion takes place. This second reactor operates at a higher pressure than the first reactor with an injection of high-pressure steam which causes a sudden expansion of the biomass (steam explosion).

When a treatment requires a pressure step (impregnation, pretreatment of cooking type or the like), it is necessary to make use of solid biomass introduction means that are compatible with these pressure steps. This is the case, for example, of compression screws, one embodiment of which is described in patent U.S. Pat. No. 4,599,138.

Patent FR 3 075 203 describes a process involving impregnation of biomass with an acidic liquor, followed by cooking and steam explosion of the impregnated biomass, with adjustment of the acidity of the acidic liquor and recycling thereof. Patent FR 3 075 201 also describes a process for pretreating biomass by acid impregnation followed by steam explosion, along with washing of the reactor feed means and recycling of the washing water into the process.

These various types of process, starting from lignocellulosic biomass, generate solid lignin-based residues, in particular after enzymatic hydrolysis, and/or after fermentation when continuing the conversion of the sugars to alcohol in particular. These solid residues, which will be referred to in the present text as “unconverted solid residue” or “lignin cake”, are rich in lignin, which does not react, or barely reacts, to the action of the enzymes used for hydrolysis of the biomass and which are generally cellulases and hemicellulases. These woody residues can be used, in particular as fuel, or can be incorporated as a filler in resin-based or bitumen-based products, for example. However, these unconverted residues do not consist (apart from a certain amount of water) solely of lignin: they may also contain hemicellulose and/or cellulose that have not reacted in the hydrolysis. And removing these residues from the biomass conversion process means losing these fractions of unreacted polymeric sugars, which impacts the biomass to sugar (or alcohol) conversion efficiency.

It has already been proposed to reuse these types of solid residues in the biomass treatment process: patent EP 2 516 661 proposes a biomass treatment process with a pretreatment of the biomass in an alkaline medium, an enzymatic hydrolysis, a fermentation of the hydrolysate obtained in order to obtain a fermentation must containing an alcohol, a separation/purification of the alcohol, and a separation of a residue cake. This residue cake is sent to a separate cellulose regeneration reactor where the cake is mixed with an alkaline solution and heated before being recycled downstream of the pretreatment. This solution is advantageous because it chooses to recover the remaining cellulose contained in this woody cake, to reincorporate it into the process, in order to increase the overall biomass conversion efficiency. However, it necessitates a separate treatment of this cake, with an additional reactor which is dedicated thereto and which will carry out an alkaline cooking of the cake before recycling it, which increases the installation and operating costs of the process as a whole, and which increases the complexity of its implementation.

Another solution has been proposed in patent EP 2 430 171, which is quite similar to the preceding one, but providing an acidic, not alkaline, cooking of the woody residue, with the same drawbacks.

The aim of the invention is thus to overcome these drawbacks. The aim of the invention is to improve the lignocellulosic biomass treatment. More particularly, the invention aims to increase biomass conversion yields, in particular in a more efficient manner than in the previous solutions.

### SUMMARY OF THE INVENTION

The subject of the invention is firstly a process for treating a lignocellulosic biomass, said process comprising:
a) a step of impregnating the biomass with a liquor, in particular an acid liquor, to obtain an impregnated biomass,
b) a step of cooking the impregnated biomass, optionally accompanied by a steam explosion, to obtain a pretreated biomass,
c) a step of enzymatic hydrolysis of the pretreated biomass, to obtain a hydrolyzed biomass in the form of sugar(s), said process also comprises
d) a step of solid/liquid separation of the hydrolyzed biomass in the form of sugar(s) or of the hydrolyzed biomass in the form of sugar(s) subsequently treated in one or more other steps after the enzymatic hydrolysis step c), in order to obtain a separated juice and an unconverted solid residue,
e) a step of recycling at least one portion of said unconverted solid residue to the impregnation step a) and/or to the cooking step b).

For the purposes of the present invention, an unconverted “solid” residue is understood to mean a residue which comprises at least 20% by weight of solids, in particular at least 30% or 35% by weight of solids. The solids content can be measured by its dry matter (DM) content, which is measured according to the standard ASTM E1756-08 (2015) “Standard Test Method for Determination of Total Solids in Biomass”. The DM of the unconverted “solid” residue according to the invention is preferably at least 20%, 30% or 35%.

The impregnating liquor may comprise a chemical compound such as an acid, a base or an oxidizing agent or by a water-based liquor, with autohydrolysis of the biomass naturally releasing an acid, notably acetic acid.

The invention therefore chooses to recycle the unconverted solid residue obtained by solid/liquid separation, either after enzymatic hydrolysis (separation from sugar juice) or after fermentation (separation from alcoholic juice), by reintroducing it into the impregnation step or the cooking step constituting the pretreatment of the biomass before the enzymatic hydrolysis thereof.

What is specific to the present invention is that the unconverted solid residue can be reintroduced directly into a pretreatment step, without a specific impregnation/cooking operation dedicated to this residue: thus, a sizeable increase in the yield (of sugar or alcohol) is obtained, without having to invest in additional energy-consuming equipment of the cooking reactor type.

Surprisingly, it has thus turned out that reintroducing this residue into the impregnation or cooking device, with the biomass being treated, made it possible to:
extract from this residue some at least of the cellulose and/or hemicellulose retained therein, the enzymatic hydrolysis reaction not enabling the complete conversion of these compounds,
and to convert them at least in part by making them once more follow the path of the “fresh” biomass in its pretreatment and then enzymatic hydrolysis steps, which, in fact, ultimately increases the production of sugars (or alcohol) of the process.

What is also surprising is that the addition of this residue to the rest of the biomass being treated did not in fact raise any problems in the operation of the pretreatment tools: whether this residue is added to the biomass in the impregnation step or in the cooking step, it did not complicate the proper functioning of the devices used, whereas it might have been feared that the addition of this residue, having a texture very different from that of biomass, would lead to problems of fouling of the devices, problems of entraining the residue from one device to another or within a device, etc. That was not the case.

Another very advantageous effect of the invention is that the final unconverted residue obtained at the end of production is depleted in cellulose/hemicellulose and enriched in lignin compared to a residue that has not undergone recycling according to the invention: this final residue thus has a higher heating value, making it more efficient as a fuel for generating heat via a step of combustion of this final residue. The heat produced can be used in the process in one or more steps requiring heating of a fluid (impregnation liquor), a reactor (cooking during pretreatment) or a reboiler of a distillation column (to purify an alcohol obtained by fermentation, etc.).

According to one embodiment, the process according to the invention targets the production of sugar juice only, the separation step d) being carried out on the sugar juice at the outlet of the enzymatic hydrolysis.

According to another embodiment, the process aims to transform all or some of the sugar juice obtained by enzymatic hydrolysis. It may then also include:—a step f) of fermenting the hydrolyzed biomass in the form of sugar(s), in order to obtain a fermented biomass comprising at least one alcohol, the solid/liquid separation step d) being carried out on said fermented biomass.

In the latter mode, it is possible, according to the invention, to recycle both the unconverted residue at the end of the enzymatic hydrolysis and at the end of the fermentation:

The enzymatic hydrolysis step c) and fermentation step f) can be carried out simultaneously on the pretreated biomass, it is then referred to as SFF, for simultaneous saccharification and fermentation, or SSCF, for simultaneous saccharification and co-fermentation. They can also be carried out one after the other, in particular in separate reactors.

The process according to the invention may also comprise:—a step g) of separation or purification, in particular of distillation, of the fermented biomass, the solid/liquid separation step d) being carried out before or after said separation or purification step g). Carrying out the separation d) before step g), when step g) is a distillation, is advantageous, since the introduction of a liquid phase containing this residue, capable of fouling the column or at the very least of hampering the operation thereof, is thus avoided. However, it may also prove advantageous to carry out the separation d) according to the invention after the separation/purification of the fermented biomass, since the solid residue may contain an alcohol fraction, and passing it through the separation/purification step may extract at least a portion of this alcohol fraction trapped in the solid residue.

As mentioned above, the process according to the invention may also include:—a step h) of combustion of the final unconverted solid residue obtained at the end of the biomass treatment, the heat produced from which is used in a step of said process requiring heating, in particular the heating of a fluid, in particular in the cooking step d) or a step g) of separation by distillation. The final residue obtained with the invention has an improved heating value, due to a lower content of cellulose/hemicellulose, which are polymeric sugars with a low heating value, unlike lignin.

Optionally, the combustion step h) may be preceded by a step i) of drying the residue, in particular so that the residue reaches a water content below a given threshold, for example less than or equal to 40% by weight, in particular less than or equal to 30% by weight.

The solid/liquid separation step d) may be carried out by filtration, in particular using a pressing or draining device, such as a filter press or a vacuum filter, a belt filter, a belt press or a centrifuging, decanting or dewatering device or the combination of various devices.

Advantageously, step a) of impregnating the biomass with a liquor and step b) of cooking the impregnated biomass can be carried out by reactors each provided with at least one biomass feed device, and step e) of recycling the unconverted solid residue is then carried out by introducing said residue with the biomass being treated into said feed device(s).

One of the feed devices at least may thus be a feed screw, which is notably at least partly conical, comprising a cowling equipped with a cage equipped with openings which allow the extraction of the solid-liquid residue from the biomass and the circulation of a washing fluid. This compression screw (also referred to as “plug screw”), as is known, creates a hermetic plug of biomass in the downstream portion of the screw, which creates a compression on the biomass which is reflected by a pressure difference between the biomass inlet and the biomass outlet of the screw. But any other known device can be used. For the impregnation step, it is possible to use a reactor operating continuously or in batch mode, or means other than a dedicated reactor, in particular a conveyor belt that is sprayed with liquor, etc.

The unconverted solid residue obtained in the solid/liquid separation step d) contains, in addition to water, a major compound in the form of lignin, and minor compounds including cellulose and/or hemicellulose and optionally one or more ethanol-type alcohols when the solid/liquid separation d) is carried out after a fermentation step f).

The unconverted solid residue obtained in the solid/liquid separation step d) may contain, by way of example, between 40% and 70% by weight of water, in particular between 50% and 60% by weight of water, between 2% and 35% DM of cellulose, in particular between 5% and 20% DM of cellulose, and between 0% and 15% DM of hemicellulose, in particular between 1% and 10% DM of hemicellulose. It may also include other minor compounds, such as ash. The various lignin, cellulose and hemicellulose contents can vary, notably according to the type of biomass used and depending on its reactivity during the enzymatic hydrolysis in particular.

Step e) of recycling the unconverted solid residue decreases its content, in the final solid residue obtained at the end of the biomass treatment, of at least one of its cellulose and hemicellulose minor compounds and increases its heating value, which is beneficial both for the biomass conversion efficiency and for the thermal integration of the process.

The dry basis composition of the final solid residue obtained at the end of the biomass treatment generally comprises mainly lignin, and less than 20% DM of cellulose and less than 8% DM of hemicellulose.

The treatment process targeted by the invention may convert the lignocellulosic biomass into sugar juice, in particular C5 and C6 juices, after enzymatic hydrolysis, or into alcohol(s), notably ethanol, after fermentation of said sugar juice.

The invention also relates to any plant implementing the process described above.

Another subject of the invention is a plant, notably for implementing the process described above, and which comprises:
a) a device, in particular a reactor, for impregnating the biomass with a liquor, in particular an acid liquor, to obtain an impregnated biomass,
b) a reactor for cooking the impregnated biomass, optionally accompanied by a steam explosion, to obtain a pretreated biomass,
c) a reactor for enzymatic hydrolysis of the pretreated biomass, to obtain a hydrolyzed biomass, said plant also comprising:
d) a device for solid/liquid separation of the hydrolyzed biomass or of the hydrolyzed biomass subsequently treated in one or more other reactors or devices positioned downstream of the enzymatic hydrolysis reactor c), in order to obtain an unconverted solid residue,
e) means for recycling at least one portion of said unconverted solid residue to the impregnation reactor a) and/or to the cooking reactor b).

The means for recycling the unconverted solid residue are conventional, and may include any suitable conveying system (e.g. by conveyor, screw, hopper or belt).

The plant according to the invention advantageously comprises an impregnation reactor a) and a cooking reactor b), which are provided with feed devices, and recycling means comprising fluid connection means between the solid/liquid separation device and at least one of said feed devices for conveying the unconverted solid residue from the separation device to the or at least one of the feed devices. The introduction of the unconverted solid residue and of the biomass being treated into the feed device (or directly into the reactor concerned) may be done jointly or through different feed points, or not simultaneously.

Another subject of the invention is the use of the process or of the plant described above for the treatment of lignocellulosic biomasses, such as wood, straw, agricultural residues, paper-making residues, and all dedicated energy crops, notably annual or perennial plants such as miscanthus, with a view to producing sugars, alcohol-type biofuels or biobased molecules.

The invention will be described below in greater detail with the aid of the figures and non-limiting examples.

### LIST OF THE FIGURES

FIG. 1 is a schematic representation of a first variant of a plant for converting lignocellulosic biomass where the process according to the invention may be applied.

FIG. 2 is a schematic representation of a second variant of a plant for converting lignocellulosic biomass where the process according to the invention may be applied.

FIG. 3 is a schematic representation of a third variant of a plant for converting lignocellulosic biomass where the process according to the invention may be applied.

FIG. 4 is a schematic representation of a plant for converting lignocellulosic biomass applying the invention.

It should be noted that the same references relate to the same stream or the same device from one figure to another.

The description of the references is presented below:
1: Entry of water into the liquor preparation tank
2: Entry of acid into the liquor preparation tank
3: Liquor preparation tool (tank)
4: Acidic liquor to impregnation tool (reactor)
5: Ground biomass
6: Impregnation tool feed device
7: Water for washing the plug-screw feeder of the impregnation tool
8: Washing liquid outlet of the plug-screw feeder 6 of the impregnation tool
9: Impregnation tool (reactor)
10: Impregnated and drained biomass
11: Pretreatment tool feed device
12: Water for washing the plug-screw feeder of the pretreatment tool
13: Pressate from the plug-screw feeder of the pretreatment tool
14: Pretreatment cooking tool (explosion reactor)
15: Injection of steam for the pretreatment
16: Pretreated biomass and steam
17: Tool (cyclone) for separating steam and pretreated biomass
18: Steam
19: Pretreated biomass
20: Enzymatic hydrolysis reactor
21: Hydrolysate containing sugars
22: Alcoholic (ethanolic) fermentation reactor
23: Fermentation wine containing ethanol (alcohol)
24: Ethanol recovery device, for example distillation column(s)
25: Concentrated alcohol
27: Solid (lignin)/liquid separation, e.g. a filter press
28: Liquid residue (vinasses)
29: Unconverted solid residue (lignin cake)
30: Clarified (solid-free) fermentation wine
31: Clarified (solid-free) hydrolyzate

### DESCRIPTION OF THE EMBODIMENTS

The invention proposes to recycle the unconverted solid residue from the process for converting lignocellulosic biomass into sugars or alcohols. This residue is mainly composed of lignin, a compound that is not converted during the process. This residue is usually separated in order to be burned in conventional biorefineries, the lignin can also be used as a compound of interest for specialty products (resins, bitumen, etc.). The invention proposes to recycle this compound upstream in the process, because the extracted lignin is not pure: it contains a fraction of polymeric sugars (cellulose and/or hemicellulose) that is not converted. Recycling these sugar polymers then makes it possible to increase the yield of the process.

The invention relates to the recycling of at least one portion of a solid residue, referred to in the present text as “lignin cake” or “unconverted solid residue”. This solid is advantageously recycled to the step of pretreating, under normal (e.g. acidic) conditions, the lignocellulosic biomass in order to increase its reactivity in the downstream enzymatic hydrolysis step. Its direct recycling to a step such as the enzymatic hydrolysis or SSCF is less relevant technically, as the access of the enzymes to the cellulose will not be improved, or not enough for the biomass conversion efficiency to be significantly improved and/or for the final residue to have a heating value that is significantly improved compared to without recycling, or at the very least much less than with the invention.

Lignin is one of the major compounds of lignocellulosic biomass (up to 30% DM). In a lignocellulosic biomass conversion process that upgrades the sugars (cellulose and hemicellulose), the lignin cake is a co-product of the process that is composed of water (water can represent 50% to 60% by weight of the cake) and solids from the biomass that are not converted during the process, namely lignin mainly, but also cellulose and hemicellulose.

Thus, table 1 below indicates the flow rate entering the process and the composition of a typical example of biomass (wheat straw) to be treated and of a lignin cake obtained after separation at the end of the alcohol production line:

TABLE 1

| | | | Unconverted |
|---|---|---|---|
| | | Biomass | solid |
| Flow rate | kg/h | 100 | 67 |
| Water | wt % | 10 | 60 |
| Cellulose | wt % | 34 | 8 |
| Hemicellulose | wt % | 27 | 3 |
| Lignin | wt % | 15 | 22 |
| Others | wt % | 14 | 7 |

And table 2 below indicates, for the same biomass and the same lignin cake, their flow rates and composition of the solid compounds, expressed here as content of dry matter, DM:

TABLE 2

| | | | Unconverted |
|---|---|---|---|
| | | Biomass | solid |
| Flow rate | kg/h | 100 | 67 |
| DM | wt % | 90 | 40 |
| Cellulose | % DM | 38 | 20 |
| Hemicellulose | % DM | 30 | 8 |
| Lignin | % DM | 17 | 55 |
| Others | % DM | 16 | 17 |

It can be seen from these tables that two unconverted sugar polymers (cellulose and hemicellulose) are present in the lignin cake, for a total of 11 wt % (or 28% DM). The invention recycles at least a portion of the lignin cake (unconverted solid) to convert these polymers and thus increase the yield of the process, since 11% of the polymeric sugars of the biomass are in the unconverted solid, without adding a treatment step dedicated to this lignin cake.

In addition, the heating value of the lignin cake (measured by the higher heating value HHV of the unconverted solid residue) increases if the sugar polymers it contains are converted. Specifically, the sugar polymers contain oxygen molecules that reduce the average HHV of the solid.

Examples of operating conditions for key steps of the biomass treatment are briefly summarized below:

### The Process

Below is a more detailed description of the various key steps of a biomass conversion process using such a plant and to which the invention may advantageously be applied: (this is one example, to which the invention is not limited).

### Step of Conditioning the Lignocellulosic Biomass

The treatment process comprises, in its first step, a step of conditioning the lignocellulosic biomass with at least one grinding so as to obtain biomass particles having a size of at most 300 mm. It is of course possible to carry out several successive grinding steps in order to reach the targeted particle size. In general, the ground biomass has a particle size (the largest size) of at most 300 mm, usually of at least 1 mm, and often between 2 and 200 mm. Any method known to those skilled in the art can be used to carry out this step. Usually, straw is ground with screens of from 5 to 100 mm. With regard to wood, it is generally chipped into parallelepipedal chips with a length of between 20 and 160 mm, a width of between 10 and 100 mm and a thickness of between 2 and 20 mm. The ground lignocellulosic biomass is conveyed to the next step by any means known to those skilled in the art, in particular a screw conveyor.

Step of Impregnation with an Acid Liquor

The treatment process according to the invention comprises a step a) of impregnating the lignocellulosic substrate with an acid liquor, so as to obtain an impregnated lignocellulosic substrate, the pH of which is from 0.1 to 3. This step aims to prepare the lignocellulosic substrate for the pretreatment step.

The impregnation is carried out in an impregnation reactor at a temperature between 10 °C and 90 °C, and preferably at atmospheric pressure. The residence time of the lignocellulosic substrate in the impregnation reactor is usually from 10 seconds to 180 minutes, preferably between 30 seconds and 60 minutes, and more preferentially still between 30 seconds and 15 minutes. Preferably, the impregnation step is performed in a single step.

The impregnation reactor or impregnator is equipped with one or more screws which transfer the lignocellulosic substrate from its inlet to the outlet opening. The impregnator is moreover equipped with one or more lines for conveying the acid liquor and also, if need be, one or more lines for withdrawing acid liquor. Said acid liquor inlet and outlet lines are generally installed so as to function by cocurrent or counter-current recycling.

The acid liquor is an aqueous solution of a strong acid, which is for example chosen from sulfuric acid, hydrochloric acid and nitric acid, for example at an acid content of between 0.5% and 4% by weight.

Step of solid/liquid separation on the lignocellulosic substrate impregnated with acid liquor The lignocellulosic substrate impregnated with acid liquor is subjected to a solid/liquid separation step in order to obtain a lignocellulosic substrate having a dry matter content of between 15% and 70% by weight and a used acid liquor. Preferably, the lignocellulosic substrate impregnated with acid liquor is first drained, in order to extract at least a portion of the free acid liquor, before being treated by solid/liquid separation.

The solid/liquid separation step may use any technique known to those skilled in the art, which may be for example decantation, centrifugation or pressing.

Preferably, pressing of the lignocellulosic substrate is carried out concomitantly with its transfer to the pretreatment step, when the latter carries out the steam explosion process which is described below. This method of carrying out the step is for example performed by a compression screw referred to as a “plug screw feeder”, the operation of which has already been described above. The formation of a plug of pressed lignocellulosic substrate ensures the pressure-tightness of the steam explosion reactor, thus preventing dangerous escapes of steam. The screw conveyor is also provided with one or more lines for withdrawing the used liquor (referred to as pressate) separated during the pressing. The pressate can be recycled to the impregnation step and/or to the step of washing with the washing liquid 12 passing through the feed screw 11.

The wet biomass obtained at the end of the solid/liquid separation step, which can be denoted by the term “washed and acidified lignocellulosic substrate” has a dry matter content preferably between 15% and 70% by weight, and more preferentially between 40% and 65% by weight.

### Step of Pretreating the Washed and Acidified Lignocellulosic Substrate

The washed and acidified lignocellulosic substrate undergoes a pretreatment step.

Cellulose (and optionally hemicelluloses) which are the targets of the enzymatic hydrolysis are not directly accessible to the enzymes. This is the reason why a pretreatment of the biomass is carried out before the enzymatic hydrolysis step. The pretreatment is notably directed toward modifying the physical and physicochemical properties of the cellulosic fraction, such as its degree of polymerization and its state of crystallinity.

Various types of pretreatment are known to those skilled in the art, they combine a chemical treatment and a heat treatment. Mention may in particular be made of acidic or basic cooking, the Organosolv process, treatments with ionic liquids and the steam explosion process.

The preferred pretreatment process is steam explosion (or “SteamEx”) carried out in an acidic medium. This is a process in which the lignocellulosic substrate is rapidly brought to a high temperature by injecting pressurized steam. Stoppage of the treatment takes place by abrupt decompression.

The operating conditions of the steam explosion process are as follows: steam is injected directly into the reactor; the temperature of the reactor is generally between 150 °C and 220 °C, preferably between 170 °C and 210 °C; the pressure is between 5 and 25 bar absolute (0.5 and 2.5 MPa), more preferentially between 8 and 19 bar absolute (0.8 to 1.9 MPa); the residence time before the expansion phase ranges from 10 seconds to 50 minutes, and preferably between 3 minutes and 30 or 40 minutes.

The steam explosion may be performed in batch or continuous mode and the depressurization step which permits destructuring of the biomass may proceed in one or more steps.

At the end of the steam explosion pretreatment step, a pretreated lignocellulosic substrate with a high dry matter content, generally between 20% and 70% by weight, and a vapor phase which may be condensed, are obtained.

Following the steam explosion under acidic conditions, the pretreated lignocellulosic substrate generally has a pH lower than that which is compatible with the medium for enzymatic hydrolysis. Thus, the lignocellulosic substrate is subjected to a neutralization step to bring its pH to a value between 4 and 6.

For the neutralization step, an aqueous solution is used that contains a neutralizing agent which can be chosen from all weak or strong bases known to those skilled in the art. The term base denotes any chemical species which, when it is added to water, gives an aqueous solution with a pH of greater than 7. Preferably, the neutralizing agent is chosen from potassium hydroxide, sodium hydroxide, aqueous ammonia and lime. More preferably still, the neutralizing agent is chosen from potassium hydroxide and aqueous ammonia, alone or in combination with one another. Preferably, the neutralizing agent is used in aqueous solution, with a weight concentration of between 2% and 75%, and more preferably still between 20% and 70%.

Neutralization is carried out at a temperature between 15 °C and 95 °C, and preferably between 20 °C and 70 °C. In general, the temperature of the neutralization step is not precisely controlled and is simply governed by the heat given off by the acid-base neutralization reaction.

The neutralization step can be carried out continuously, in batch mode or in fed-batch mode.

It should be noted that an optional washing step can be carried out before or after the neutralization step, on all or part of the pretreated lignocellulosic substrate.

If a washing is applied, a liquid stream is brought into contact with the pretreated lignocellulosic substrate, then the liquid is separated from the solid. The washing step can be carried out by percolation, by successive mixing and liquid/solid separation operations, by washing on a belt filter or by any other technique known to those skilled in the art. The washing liquid used can be water or a process stream. The weight ratio between the washing liquid added and the liquid contained in the substrate to be washed is generally between 0.5 and 4. The washing step generates a sugary washing liquor containing a portion of the hemicelluloses solubilized during the pretreatment. This washing liquor can for example be used as a source of carbon for the production of biocatalysts (enzymes and/or microorganisms). The washing step is generally carried out at a temperature between 10 °C and 95 °C.

### Enzymatic Hydrolysis Step

The pretreated lignocellulosic substrate, optionally neutralized and washed, is sent to the enzymatic hydrolysis step of the process.

The pretreated lignocellulosic substrate which is sent to the enzymatic hydrolysis step has a dry matter content generally of between 15% and 70% by weight.

The objective of the enzymatic hydrolysis is to hydrolyze (depolymerize), by means of biocatalysts, the hemicelluloses and cellulose into fermentable sugars, preferably glucose.

The enzymatic hydrolysis step is carried out under mild conditions, at a temperature of the order of 40 °C and 55 °C, preferably between 45 °C and 50 °C and at a pH of from 4.0 to 5.5, and more preferentially still between 4.5 and 5.2. The dry matter content of the enzymatic hydrolysis medium is between 2% and 45% by weight, preferably between 10% and 30% by weight. It is carried out by means of enzymes produced by a microorganism. Natural or genetically modified microorganisms, such as the fungi of the genera Trichoderma, Aspergillus, Penicillium or Schizophyllum, or anaerobic bacteria of, for example, the genus Clostridium, produce a cocktail of enzymes containing in particular cellulases and hemicellulases, suitable for extensive hydrolysis of cellulose and hemicelluloses. The enzymatic hydrolysis can be carried out continuously or in batch mode or in fed-batch mode, in one or more reactors. The residence time is between 5 hours and 200 hours and preferably between 24 hours and 120 hours and more preferentially still between 48 hours and 120 hours.

At the end of the step, a hydrolyzate containing fermentable sugars is recovered from the bioreactor, which hydrolyzate is then treated in the fermentation step.

It should be noted that the hydrolyzate obtained can optionally undergo one or more treatment steps before the fermentation step. For example, there may be a return to the pH, a partial purification with a view to limiting the content of inhibitor compound for the fermentation microorganism, or an at least partial separation of the solid residues contained in the hydrolyzate (and to thus obtain the unconverted solid residue to be treated according to the invention).

Step of Fermenting the Hydrolyzate, when it is Desired to Continue the Conversion of the Sugars Obtained to Alcohol(s)

According to the step of the process for producing solvents and/or alcohols, the optionally treated hydrolyzate is sent to the fermentation step enabling the conversion, by means of one or more microorganisms of different genera, of the fermentable sugars into solvent and/or alcohols of interest. The fermentation methods are known to those skilled in the art and are described in particular in document U.S. Pat. No. 8,456,633.

The term “solvent” is intended to denote organic compounds other than alcohols, for example organic compounds having a ketone function such as acetone.

The term “alcohol” denotes in particular ethanol, propanol, isopropanol and butanol.

The natural or genetically modified microorganisms can be chosen, for example, from Saccharomyces cerevisiae, Schizosaccharomyces pombe, Saccharomyces uvarum, Saccharomyces diastaticus, Kluyveromyces fragilis, Candida shehatae, Pichia stipitis, Pachysolen tannophilus or the bacteria Zymomonas mobilis, Clostridium acetobutylicum, Escherichia coli.

In the context of the invention, the fermentation step makes it possible, for example, to produce ethanol alone or as a mixture with butanol, propanol, isopropanol and/or acetone. For example, the fermentation microorganism may be capable of producing an “ABE (acetone-butanol-ethanol)” mixture or else an “IBE (isopropanol-butanol-ethanol)” mixture.

Preferably, the microorganism chosen is a natural or genetically modified yeast of the genus Saccharomyces capable of producing ethanol.

At the end of the step, a fermentation must, diluted in products of interest, is recovered.

According to one embodiment of the process, the hydrolysis and fermentation steps may be carried out at the same time, in at least one same bioreactor, so that the enzymatic hydrolysis and the fermentation are carried out simultaneously according to a process denoted by the term “simultaneous saccharification and fermentation (SSF)”. When the hydrolysis step is merged with the fermentation step, the operating conditions, in particular temperature conditions, can be adapted to be compatible with the tolerances of the fermentation microorganism. For example, the temperature can be lowered between 28 °C and 45 °C, and preferably between 30 °C and 35 °C, when the fermentation is carried out with a yeast of the genus Saccharomyces. The pH is preferably adjusted between 5 and 5.5 in order to promote the performance of the yeasts.

The production unit carrying out the process according to the invention may comprise, in addition to the plants already described, units for the in situ production of enzymes and/or yeasts.

Step of Separating the Solvents and/or Alcohols from the Fermentation Must

The process according to the invention finally comprises a step of separating the product(s) of interest from the fermentation must, which is preceded or followed by a solid/liquid separation step in order to remove at least one fraction of the solid matter contained in the fermentation must, and to produce the unconverted solid residue which will be treated according to the invention.

Preferably the step of separating the product(s) of interest, for example ethanol, uses one or more distillations, which is technology well known to those skilled in the art.

The feedstock: the lignocellulosic biomass. In accordance with the invention, the feedstock of the process may be a biomass alone or as a mixture. The amount of water contained in the crude feedstock is generally at least 10%, notably between 10% and 70% by weight.

The crude biomass is chosen from any type of biomass, preferably biomass of solid type, and in particular biomass of lignocellulosic type. Nonlimiting examples of biomass types concern, for example, farming residues (notably straw, corn cobs), forestry management residues, forestry management products, wood mill residues and dedicated crops, for example short-rotation coppice. Preferably, the crude biomass, also referred to as native biomass, is lignocellulosic biomass. It essentially comprises three natural constituents present in variable amounts depending on its origin: cellulose, hemicellulose and lignin.

The lignocellulosic biomass feedstock is preferably used in its crude form, i.e. containing all of these three cellulose, hemicellulose and lignin constituents.

In one preferred embodiment of the invention, the lignocellulosic biomass is chosen from grassy biomass, farming residues such as straw waste, corn cobs, sugarcane bagasse, forestry management residues or wood mill residues such as wood chips or any other type of woody residues.

The impregnation fluid: The optional fluid, injected for the impregnation, is an aqueous liquid solution optionally containing acid, at a temperature between 10 °C and 95 °C and at atmospheric pressure. The pH of this chemical solution is between 0.1 and 12.0, preferably between 0.1 and 7, preferably between 0.3 and 2. According to a preferred embodiment, the liquor used is an acid catalysis liquor, and the pH of the liquor is adjusted to between 0.1 and 4, notably between 0.3 and 2. Examples of acids that may be used include at least one acid chosen from sulfuric acid, hydrochloric acid, nitric acid and oxalic acid. Their content, in the aqueous phase, is preferably between 0.2% and 8% by weight.

The invention applies in an analogous manner to different processes and plants, notably to:—plants which are intended only for the production of sugars, and which therefore do not provide alcoholic fermentation,—plants which provide pretreatment by cooking without prior impregnation with a liquor (e.g. autohydrolysis),—plants which provide a pretreatment by cooking with a prior impregnation with a non-acidic liquor.

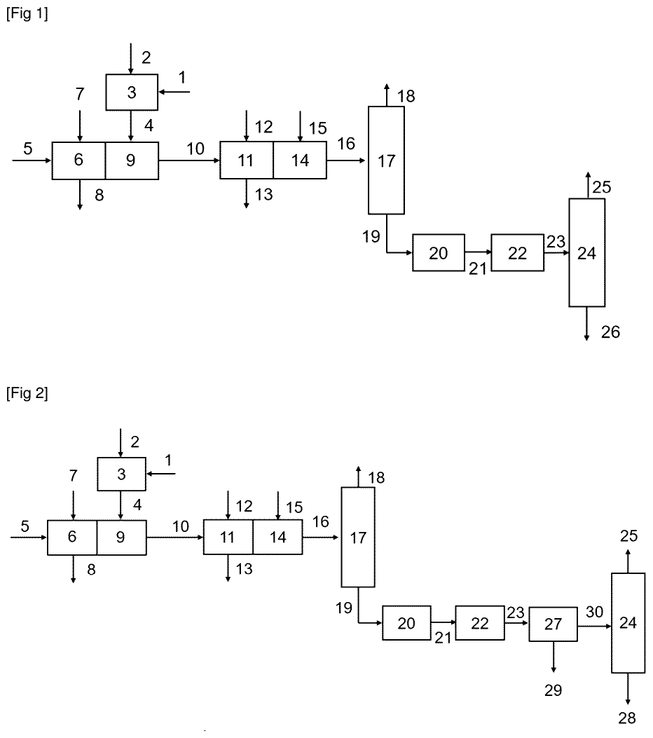

FIG. 1 very schematically represents a nonlimiting variant of a plant for converting lignocellulosic biomass wherein the invention can be applied, which plant makes provision for a pretreatment of the biomass including an impregnation with an acidic liquor, then a cooking/steam explosion, then an enzymatic hydrolysis followed by an alcoholic fermentation, in order to convert the biomass to ethanol.

FIG. 1 therefore represents a biomass conversion carried out in the following manner: the ground biomass 5 (optionally impregnated with a catalytic liquor) is introduced into an impregnation reactor 9 via a feed device 6. This may be a pressurized feed means, for example a screw, also referred to as a plug screw feeder, the end portion of which is conical, which has a cowling with a drainage screen, a washing water inlet 7 and a washing water outlet 8. A hermetic plug of biomass is created in the downstream portion of the screw, which creates a compression on the biomass which is reflected by a pressure difference between the biomass inlet and the biomass outlet of the screw of at least 0.05 MPa. The compression applied to the biomass may lead to the expulsion of a portion of the liquid contained in the biomass, notably when the DM of the biomass is less than 80% before it enters the pressurized feed means 6. The liquid thus extracted mixes with the washing water and is drawn off with the used washing water 8. The impregnation reactor is also supplied with acidic liquor 4 (water supplemented with sulfuric acid) originating from a liquor preparation tank 3, itself supplied with acid 2 and with water 1. The impregnated and drained biomass 10 leaves the reactor 9 in order to feed a steam explosion cooking reactor 14 via another feed device 11, for example of compression type, like the feed device 6. In this device 11, owing to the compression exerted on the biomass, a liquid 13 resulting from this pressing, also referred to as pressate, is recovered, which is composed of water and acid. The device 11 is washed, using a dedicated inlet, with a washing liquid 12 (water and/or a recycled liquid, as seen below), which is then discharged via the outlet through which the pressate 13 is also discharged.

The reactor 14 is also supplied with steam 15. On leaving the reactor 14, the biomass-steam mixture passes into a tool 17 for separating the biomass 19 and the steam 18. The biomass 19 is then treated in an enzymatic hydrolysis reactor 20, then once hydrolyzed to sugars, the hydrolyzed biomass 21 (also referred to as hydrolysis must) passes into an alcoholic fermentation reactor 22. The biomass fermented to alcohol 23 (also referred to as fermentation must) is then conveyed into one or more distillation columns 24 to obtain concentrated alcohol 25 and crude vinasses 26, which are solid/liquid residues, as a mixture or separated depending on the arrangement of the columns 24. A solid/liquid separation is carried out by a device 27 of the filter press type, and a liquid residue 28 (the vinasses) and a residue 29 which is the lignin cake of interest to the invention are obtained at the outlet.

This is only one example of a plant, which may also have numerous variants. Thus, the enzymatic hydrolysis and fermentation may be carried out together in the same reactor, it is then referred to as SSCF (simultaneous saccharification and co-fermentation).

FIG. 2 is a variant of the process according to FIG. 1, where, all other things being equal, the solid/liquid separation by the filter press 27 is performed on the fermentation wine 23 before the distillation column(s) 24: the lignin cake 29 is extracted, and the liquid residue, that is to say the fermented wine without solid residue, 30 is then conveyed into the distillation column(s) 24.

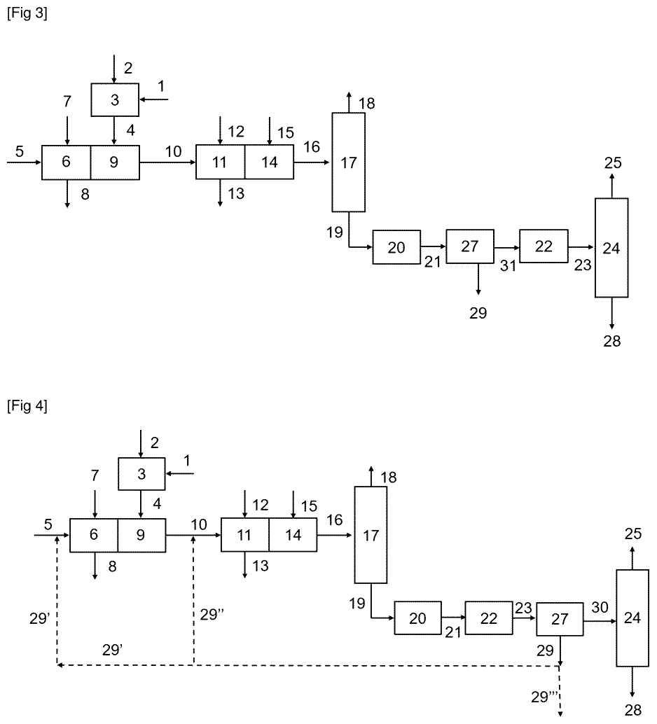

FIG. 3 is a variant of the process according to FIG. 2, where, all other things being equal, the solid/liquid separation device 27 of the filter press type is arranged between the enzymatic hydrolysis reactor 20 and the fermentation reactor 22: here the separation is carried out on the hydrolyzate 21 which is not yet fermented, and the liquid part of the hydrolyzate (sugar juice) 31 then goes into the fermentation reactor 22. In a variant (not shown) of the process according to FIG. 3, only the production of sugar juice 31 is targeted, the conversion of which by fermentation is not continued on the same production line.

In all these variants, the objective of the invention is to treat the lignin cake 29, which is ultimately intended to be burned to produce energy.

FIG. 4 shows two alternative or cumulative implementations of the invention, starting from the variant of FIG. 1:—the lignin cake 29 is recycled at least in part (stream 29′) to the feed device 6 of the impregnation reactor 9. This recycling before impregnation is advantageous because the lignin cake 29 will be brought back into contact with the (acidic) impregnation liquor in the reactor 9, which will help to extract the residual polymeric sugars contained in the cake and subsequently make them react more easily during the enzymatic hydrolysis. It might have been feared that the lignin cake would crumble and settle at the bottom of the reactor 9, without managing to be conveyed with the biomass, but surprisingly this was not the case, even when the biomass is conveyed into the reactor from its injection point to its outlet point by screws inside the reactor;
the lignin cake is recycled at least in part (stream 29″) to the feed device 11 of the cooking/steam explosion reactor 14. In this case, the lignin cake is not impregnated with liquor again, but it is entrained by the biomass that is impregnated;
optionally, a portion of the lignin cake (stream 29′″) is not recycled: The lignin cake may be only partially recycled. At the end of production, the unrecycled cake (and the final residue) is stored to serve as fuel, either on the production line or for another use outside the production line, as fuel or to be incorporated into various products.

The same types of recycling apply similarly to the lignin cakes according to the processes of FIGS. 1 and 3.

### IMPLEMENTATION EXAMPLES

Example 1 (comparative) This example, not in accordance with the invention, separates the unconverted solid 29 without recycling it. The biomass 5 is a lignocellulosic biomass, wheat straw. Its composition is indicated in table 3 below:

TABLE 3

| | | |
|---|---|---|
| Cellulose | 34% | by weight |
| Hemicellulose | 27% | by weight |
| Lignin | 15% | by weight |
| Water | 10% | by weight |
| Others | 14% | by weight |

The biomass is treated according to the process shown in FIG. 2. After hydrolysis, the cellulose is converted into glucose or into glucose oligomers, and the hemicellulose is converted into xylose or into xylose oligomers. For this purpose, the cellulose or hemicellulose flow rates are expressed as potential glucose or potential xylose.

The term “potential sugar” (xylose, glucose for example) used below defines the addition of the various sugars, irrespective of their form: monomeric or polymeric sugar. Specifically, after pretreatment by cooking, a portion of the sugars remains in sugar polymer form (cellulose or hemicellulose for example), and a portion of the sugars is in the sugar monomer form (glucose or xylose for example). This measurement may be carried out using the standard ASTM E1758-01 (2020) “Standard Test Method for Determination of Carbohydrates in Biomass by High Performance Liquid Chromatography”. As defined in the standard, to express this amount of sugars in polymer form (cellulose for example), it is necessary to subtract the water of hydrolysis from this amount.

The operating procedure is the following: 642 kg/h of this 50 mm ground biomass 5 enter the process, i.e. 242.5 kg/h of potential glucose, 197 kg/h of potential xylose and 96.3 kg/h of lignin. Since the process is intended to produce ethanol by fermentation, these potential sugar flow rates correspond to 224.6 kg/h of potential ethanol. The ground biomass 5 enters the feed screw 6 which is washed with a flow rate of 200 kg/h of washing water 7. A first solid/liquid stream 8 of 203.8 kg/h leaves the process, in this stream are 1.3 kg/h of potential glucose, 1.1 kg/h of potential xylose and 0.5 kg/h of lignin. In the impregnation step (reactor 9), 1622.6 kg/h of water and 84.2 kg/h of sulfuric acid are added to the reactor from the preparation tank 3 and constitute the impregnation liquid 4. At the inlet of the cooking reactor 14, the transfer zone 11 is fed with the impregnated biomass 10. The transfer zone 11 (compression screw) is washed with 4087.0 kg/h of water 12, a second solid/liquid stream 13 of 5099.9 kg/h leaves zone 11. In this stream 13 are 2.7 kg/h of potential glucose, 2.2 kg/h of potential xylose and 1.1 kg/h of lignin. The cooking reactor 14 is heated by a steam stream 15 of 3471.1 kg/h. At the outlet of this reactor 14, the stream of pretreated biomass 16 of 4803.2 kg/h, comprising 237.8 kg/h of potential glucose, 155.2 kg/h of xylose and 94.7 kg/h of lignin, leaves.

This stream 16 is separated into a gaseous stream 18 of 3039.8 kg/h essentially comprising steam and a solid/liquid stream 19 of 1763.4 kg/h, comprising 237.8 kg/h of potential glucose, 155.2 kg/h of potential xylose and 94.7 kg/h of lignin. The stream 19 then undergoes an enzymatic hydrolysis step in an enzymatic hydrolysis reactor 20. After enzymatic hydrolysis, the stream 21 comprises 38.9 kg/h of potential polymeric glucose, 198.9 kg/h of glucose, 15.4 kg/h of potential polymeric xylose, 139.8 kg/h of xylose and 94.7 kg/h of lignin. The stream 21 then undergoes a fermentation step in a fermentation reactor 22.

After fermentation, the stream 23 comprises 38.9 kg/h of potential polymeric glucose, 15.4 kg/h of potential polymeric xylose, 94.7 kg/h of lignin and 157.8 kg/h of ethanol. The stream 23 then undergoes solid/liquid separation in a separation tool 27 (a filter press for example).

The solid stream resulting from the separation 27 is the stream 29, defined as the unconverted solid, comprising 38.9 kg/h of potential polymeric glucose, 15.4 kg/h of potential polymeric xylose, 94.7 kg/h of lignin and 2.2 kg/h of ethanol (loss of ethanol in the solid). The liquid stream resulting from the separation 27 is the stream 30, comprising 155.7 kg/h of ethanol. The stream 30 is then distilled in a distillation column 24, the final stream 25 then comprises 154.1 kg/h of ethanol. Compared to the ethanol potential of the biomass of 224.6 kg/h, the process has a yield of 68.6%.

Example 2 (according to the invention) This example according to the invention proposes recycling the unconverted solid 29 to the inlet of the cooking reactor 14 via the feed screw 11. The biomass used for this example is the same as for example 1. 642 kg/h of this 50 mm ground biomass 5 enter the process, i.e. 242.5 kg/h of potential glucose, 197 kg/h of potential xylose and 96.3 kg/h of lignin. Since the process is intended to produce ethanol by fermentation, these potential sugar flow rates correspond to 224.6 kg/h of potential ethanol.

The ground biomass 5 enters the feed screw 6 which is washed with a flow rate of 200 kg/h of washing water 7. A first solid/liquid stream 8 of 203.8 kg/h leaves the process, in this stream are 1.3 kg/h of potential glucose, 1.1 kg/h of potential xylose and 0.5 kg/h of lignin.

In the impregnation step (reactor 9), 1622.6 kg/h of water and 84.2 kg/h of sulfuric acid are added to the reactor from the preparation tank 3 and constitute the impregnation liquid 4.

At the inlet of the cooking reactor 14, the transfer zone 11 is fed with the impregnated biomass 10 and with the stream 29″ corresponding to 80% of the unconverted solid, and which is recycled from the solid/liquid separation 27. 20% of the unconverted solid is removed from the process.

This is because preferably not 100%, but only a fraction of the unconverted solid is recycled, for example at least 1%, 5%, 10%, 20%, 30%, 40%, 50% or even at least 60% or at least 70 or 80% by weight of the residue, so that the lignin compound can be discharged from the biomass conversion line. The invention is advantageous even when this recycling is not very high, and a very high recycling rate may result in the size of the equipment having to be increased, which may not be desirable.

The maximum amount of final solid residue corresponds in fact to the amount of lignin in the biomass.

This recycled stream 29″ comprises 31.1 kg/h of potential glucose, 12.3 kg/h of potential xylose, 75.8 kg/h of lignin and 1.7 kg/h of ethanol. The transfer zone 11 (compression screw) is washed with 4087.0 kg/h of water 12, a second solid/liquid stream 13 of 5099.9 kg/h leaves zone 11. In this stream 13 are 2.5 kg/h of potential glucose (compared to 2.7 kg/h in example 1), 1.9 kg/h of potential xylose (compared to 2.2 kg/h in example 1) and 1.6 kg/h of lignin (compared to 1.1 kg/h in example 1), that is to say that this loss of solid is equivalent to example 1 in terms of crude flow rate, but that it is less rich in potential sugars than for example 1. Specifically, the solid included in the stream 13 must pass through the holes of the cage of the screw 11, it is these holes that set the flow rate of solid of the stream 13, and not the flow rate of solid in the screw inlet stream. The cooking reactor 14 is heated by a steam stream 15 of 3471.1 kg/h. At the outlet of this reactor 14, the stream of pretreated biomass 16 of 4982.9 kg/h, comprising 268.9 kg/h of potential glucose, 165.1 kg/h of xylose and 170.5 kg/h of lignin, leaves.

This stream 16 is separated into a gaseous stream 18 of 3039.8 kg/h essentially comprising steam and a solid/liquid stream 19 of 2005.3 kg/h, comprising 269.1 kg/h of potential glucose, 165.4 kg/h of potential xylose and 170.0 kg/h of lignin. The stream 19 then undergoes an enzymatic hydrolysis step in an enzymatic hydrolysis reactor 20. After enzymatic hydrolysis, the stream 21 comprises 63.9 kg/h of potential polymeric glucose, 205.2 kg/h of glucose, 17.5 kg/h of potential polymeric xylose, 147.9 kg/h of xylose and 170 kg/h of lignin.

The stream 21 then undergoes a fermentation step in a fermentation reactor 22. After fermentation, the stream 23 comprises 63.9 kg/h of potential polymeric glucose, 17.5 kg/h of potential polymeric xylose, 170 kg/h of lignin and 164.6 kg/h of ethanol. The stream 23 then undergoes solid/liquid separation in a separation tool 27 (a filter press for example). The solid stream resulting from the separation 27 is 80% recycled as described above (stream 29″) and 20% leaves the process (stream 29″) comprising 32.8 kg/h of potential polymeric glucose, 5.1 kg/h of potential polymeric xylose, 94.2 kg/h of lignin and 0.5 kg/h of ethanol. The liquid stream resulting from the separation 27 is the stream 30, comprising 162.3 kg/h of ethanol. The stream 30 is then distilled in a distillation column 24, the final stream 25 then comprises 160.7 kg/h of ethanol.

Compared to the ethanol potential of the biomass of 224.6 kg/h, the process has a yield of 71.5%, i.e. an increase of 2.9 yield points.

The HHV of the solid residue itself increased by 2.7% (from 20.9 MJ/kg DM of lignin to 21.5 MJ/kg DM of lignin). HHV is the acronym for higher heating value, which corresponds to the total amount of heat released at constant volume by the combustion of one kg or one Nm3 of a fuel at standard atmospheric pressure, the water formed during combustion being returned to the liquid state and the other products to the gaseous state.

In conclusion, compared with the prior art, the present invention leads to the following advantages:
the overall sugar/ethanol yield of the process is improved by recycling the unconverted polymeric sugars (cellulose and hemicellulose). After recycling to the pretreatment, these polymeric sugars are partly converted into monomeric sugars or are made more accessible to the enzymes used in the enzymatic hydrolysis step;
the quality of the lignin is improved since the conversion of the sugars has made it purer/richer in lignin, the HHV of the solid is therefore increased since the polymeric sugars have a lower HHV than that of lignin.

1. A process for treating a lignocellulosic biomass, said process comprising:
a) a step of impregnating the biomass with a liquor, in particular an acid liquor, to obtain an impregnated biomass,
b) a step of cooking the impregnated biomass, optionally accompanied by a steam explosion, to obtain a pretreated biomass,
c) a step of enzymatic hydrolysis of the pretreated biomass, to obtain a hydrolyzed biomass in the form of sugar(s), characterized in that said process also comprises:
d) a step of solid/liquid separation of the hydrolyzed biomass in the form of sugar(s) or of the hydrolyzed biomass in the form of sugar(s) subsequently treated in one or more other steps after the enzymatic hydrolysis step c), in order to obtain a separated juice and an unconverted solid residue,
e) a step of recycling at least one portion of said unconverted solid residue to the impregnation step a) and/or to the cooking step b),
h) a step of combustion of the unconverted solid residue, referred to as the final solid residue, obtained at the end of the biomass treatment, the heat from which is used in a step of said process requiring heating.

2. The process as claimed in claim 1, characterized in that it also comprises:
a step f) of fermenting the hydrolyzed biomass in the form of sugar(s), in order to obtain a fermented biomass comprising at least one alcohol, and in that the solid/liquid separation step d) is carried out on said fermented biomass.

3. The process as claimed in claim 1, characterized in that the enzymatic hydrolysis step c) and fermentation step f) are carried out simultaneously on the pretreated biomass.

4. The process as claimed in claim 2, characterized in that it also comprises:
a step g) of separation or purification, in particular of distillation, of the fermented biomass, and in that the solid/liquid separation step d) is carried out before or after said separation or purification step g).

5. The process as claimed in claim 1, characterized in that the step of said process requiring heating, in particular the heating of a fluid, is the cooking step d) or a step g) of separation by distillation, said step h) optionally being preceded by a step i) of drying said residue.

6. The process as claimed in claim 1, characterized in that the solid/liquid separation step d) is carried out by filtration, in particular using a pressing or draining device, such as a filter press or a vacuum filter, a belt filter, a belt press or a centrifuging, decanting or dewatering device or the combination of various devices.

7. The process as claimed in claim 1, characterized in that step a) of impregnating the biomass with a liquor and step b) of cooking the impregnated biomass are carried out by reactors each provided with at least one biomass feed device, and in that step e) of recycling the unconverted solid residue is carried out by introducing said residue with the biomass being treated into said feed device(s).

8. The process as claimed in claim 1, characterized in that the unconverted solid residue obtained in the solid/liquid separation step d) contains between 40% and 70% by weight of water, in particular between 50% and 60% by weight of water, between 2% and 35% DM of cellulose, in particular between 5% and 20% DM of cellulose, and between 0% and 15% DM of hemicellulose, in particular between 1% and 10% DM of hemicellulose.

9. The process as claimed in claim 1, characterized in that the treatment converts the lignocellulosic biomass into sugar juice, in particular C5 and C6 juices, after enzymatic hydrolysis, or into alcohol after fermentation of said sugar juice.

10. A plant for implementing the process as claimed in claim 1, characterized in that it comprises:
a) a device, in particular a reactor, for impregnating the biomass with a liquor, in particular an acid liquor, to obtain an impregnated biomass,
b) a reactor for cooking the impregnated biomass, optionally accompanied by a steam explosion, to obtain a pretreated biomass,
c) a reactor for enzymatic hydrolysis of the pretreated biomass, to obtain a hydrolyzed biomass, characterized in that said plant also comprises:
d) a device for solid/liquid separation of the hydrolyzed biomass or of the hydrolyzed biomass subsequently treated in one or more other reactors or devices positioned downstream of the enzymatic hydrolysis reactor c), in order to obtain an unconverted solid residue,
e) means for recycling at least one portion of said unconverted solid residue to the impregnation reactor a) and/or to the cooking reactor b),
h) a reactor for combustion of the unconverted solid residue, referred to as the final solid residue, obtained at the end of the biomass treatment, the heat from which is used in a step of said process requiring heating.

11. The plant as claimed in claim 1, characterized in that the impregnation reactor a) and the cooking reactor b) are provided with feed devices, and in that the recycling means comprise fluid connection means between the solid/liquid separation device and at least one of said feed devices for conveying the unconverted solid residue from the separation device to the or at least one of the feed devices.

12. A method for producing sugars, alcohol-type biofuels or biobased molecules comprising performing the process as claimed in claim 1, wherein the lignocellulosic biomass treated is wood, straw, agricultural residues, paper-making residues, and all dedicated energy crops, notably annual or perennial plants such as miscanthus.
